(19) United States
(12) Patent Application Publication
(10) Pub. No.: US 2025/0041103 A1
(43) Pub. Date: Feb. 6, 2025

(54) APPARATUS FOR RECEIVING DISCHARGED URINE

(71) Applicant: PUREWICK CORPORATION, Covington, GA (US)

(72) Inventor: Kathleen Davis, Atlanta, GA (US)

(21) Appl. No.: 18/757,964

(22) Filed: Jun. 28, 2024

Related U.S. Application Data
(63) Continuation of application No. 17/453,260, filed on Nov. 2, 2021, now Pat. No. 12,048,644.
(60) Provisional application No. 63/109,084, filed on Nov. 3, 2020.

(51) Int. Cl. A61F 5/455; A61M 1/00

(52) U.S. Cl. CPC A61F 5/455; A61M 1/60; A61M 1/80; A61M 2205/02

(57) ABSTRACT

An example device suitable for collecting and transporting urine away from the body of a person may include a fluid impermeable housing defining an internal volume. The device can include a porous material disposed within the internal volume. The fluid impermeable housing can define an opening in fluid communication with the internal volume. The porous material includes a contact surface and first and second sidewalls. The sidewalls can extend from the contact surface, for example, substantially orthogonal to the contact surface, or laterally and inwardly from the contact surface at an acute angle.

### CROSS-REFERENCE TO RELATED APPLICATIONS

This application is a continuation of U.S. application Ser. No. 17/453,260 filed Nov. 2, 2021, which claims priority to U.S. Provisional Patent Application No. 63/109,084 filed on Nov. 3, 2020, the disclosure of each of which is incorporated herein, in its entirety, by this reference.

### BACKGROUND

In various circumstances, a person may have limited or impaired mobility such that typical urination processes are challenging or impossible. For example, a person may experience or have a disability that impairs mobility. A person may have restricted travel conditions such as those experienced by pilots, drivers, and workers in hazardous areas. Additionally, sometimes fluid collection is needed for monitoring purposes or clinical testing.

Urinary catheters, such as a Foley catheter, can be used to address some of these circumstances, such as incontinence. Unfortunately, however, urinary catheters can be uncomfortable, painful, and can lead to complications, such as infections. Additionally, bed pans, which are receptacles used for the toileting of bedridden patients, such as those in a health care facility, are sometimes used. Bed pans, however, can be prone to discomfort, spills, and other hygiene issues.

Females who suffer the most severe consequences of urinary incontinence, such as discomfort, rashes, and sores are typically elderly and often bedbound. They also require continuous assistance to maintain hygiene. These patients typically lay on their back for extended periods of time and often have difficulty reaching and manipulating fluid collection devices.

### SUMMARY

Embodiments are directed to fluid collection devices suitable for collecting and transporting urine away from the body of a person. In an embodiment of the present disclosure, a fluid collection device is disclosed. The fluid collection device can include a fluid impermeable housing defining an internal volume. The fluid impermeable housing defines an opening in fluid communication with the internal volume. The fluid collection device can include a porous material partially disposed within the internal volume and in fluid communication with the opening. The porous material includes a contact surface disposed external to the internal volume. The porous material can include a first side wall and a second side wall. In an embodiment, each of the first and second side walls can extend substantially orthogonal to the contact surface. In an embodiments, each of the first and second side walls can extend laterally and inwardly from the contact surface at an angle, such as, an acute angle. The porous material can be configured to be placed at least proximate to a urethra of a female user when the fluid collection device is disposed in a pubic region of the female user.

In another embodiment of the present disclosure, a fluid collection device is disclosed. The fluid collection device includes a fluid impermeable housing defining an internal volume. The fluid impermeable housing defines an opening in fluid communication with the internal volume. The fluid collection device includes a porous material at least partially disposed within the internal volume and in fluid communication with the opening. The porous material can include a portion extending from the internal volume. The portion includes a contact surface and first and second side walls. The first and second side walls can extend laterally and inwardly from the contact surface at an angle. At least one of the porous material or the fluid impermeable housing can place be placed proximate to a urethra of a female user when the fluid collection device is disposed in a pubic region of the female user.

In another embodiment of the present disclosure, a system for transporting urine away from a user is disclosed. The system includes a fluid collection device and a suction device. The suction device is operably coupled to the fluid collection device such that fluid can be drawn from the fluid collection device via the suction device. The fluid collection device includes a fluid impermeable housing defining an internal volume. The fluid impermeable housing defines an opening in fluid communication with the internal volume. The fluid collection device can include a porous material partially disposed within the internal volume and in fluid communication with the opening. The porous material includes a contact surface disposed external to the internal volume. The porous material can include a first side wall and a second side wall. In an embodiment, each of the first and second side walls can extend substantially orthogonal to the contact surface. In an embodiments, each of the first and second side walls can extend laterally and inwardly from the contact surface at an angle, such as, an acute angle. The porous material can be configured to be placed at least proximate to a urethra of a female user when the fluid collection device is disposed in a pubic region of the female user.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection device, according to an embodiment.

FIG. 1B is a side view of the fluid collection device of FIG. 1A.

FIG. 1C is a top view of the fluid collection device of FIG. 1A.

FIG. 1D is a cross-section view of the fluid collection device of FIG. 1A.

FIG. 2A is an example cross-section view of an embodiment of the fluid collection device taken through the section lines 2-2 of FIG. 1A.

FIG. 2B is an example cross-section view of an embodiment of the fluid collection device taken through the section lines 2-2 of FIG. 1A.

FIG. 2C is an example cross-section view of an embodiment of the fluid collection device taken through the section lines 2-2 of FIG. 1A.

FIG. 2D is an example cross-section view of an embodiment of the fluid collection device taken through the section lines 2-2 of FIG. 1A.

FIG. 3 is a block diagram of a system for fluid collection, according to an embodiment.

### DETAILED DESCRIPTION

Embodiments are directed to fluid collection devices suitable for collecting and transporting urine away from the body of a person. The embodiments of the fluid collection devices described herein may be positioned in a pubic region of a female user to collect urine or other fluids expelled by the user. In embodiments, the fluid collection device includes a fluid impermeable housing forming or defining an internal volume and an opening to the internal volume. The fluid collection device includes a fluid permeable or porous material disposed partially within the internal volume and in fluid communication with the opening. The porous material includes a contact surface in fluid communication with the internal volume. The contact surface can contact the pubic region of the female user. The porous material also includes first and second side walls. In embodiments, each of the first and second side walls can extend substantially orthogonal to the contact surface. In some embodiments, each of the first and second side walls can extend laterally and inwardly from the contact surface at an angle, such as, an acute angle.

The fluid collection device can include a fluid conduit disposed within the internal volume and in fluid communication with at least a portion of the internal volume. The fluid conduit can enable extraction of fluid from the internal volume. For example, the fluid conduit can be coupled to a vacuum source which extracts urine or other fluids from the internal volume by applying a negative pressure to the internal volume of the receptacle. One suitable non-limiting example of a vacuum source that can be used is the DryDoc Vacuum Station, available from PureWick, Inc.

In embodiments, the fluid impermeable housing can include and/or house a plurality of layers, for example, one or more fluid permeable layers, one or more absorbent layers, one or more hydrophobic layers, one or more fluid permeable layers, one or more fluid impermeable layers, and so on. For example a porous material can be positioned partially within the internal volume of the fluid impermeable housing to provide an interface with the user's genitals that is substantially moisture free.

The fluid collection device can be positioned in a pubic region of a female user. For example, the fluid collection device can be disposed between the thighs and/or buttocks of the female user and abut or be positioned adjacent to a urethral opening of the female user's genitals. A portion of the fluid collection device can abut or otherwise be disposed adjacent a perineum of the female user. While positioned in the pubic region of the female user, the fluid impermeable housing and/or porous material can be shaped or otherwise formed to more proficiently retain the fluid collection device in a fixed position relative to the user. In embodiments, at least one of the fluid permeable housing or the porous material can form a cross-section, for example, at least part of a rectangle, a square, or a trapezoid. In other words, one or more of the contact surface, the first side wall, and/or the second side wall of the porous material can be oriented relative to each other as to provide a cross-section which is more retainable by a patient using the fluid collection device. For example, the side walls may be planar and orthogonal to the contact surface to provide a surface area which engages with a user's thighs, buttocks, and/or genital region to retain the fluid collection device in a fixed position relative to the user. Additionally, or alternatively, the contact surface, the first side wall, and the second side walls can form corners or edges which engage with a user's thighs, buttocks, and/or genital region to retain the fluid collection device in a fixed position relative to the user. By incorporating a porous material defined by a plurality of walls and surfaces, the fluid collection device can be reliably retained by even small persons having relatively small thighs and buttocks.

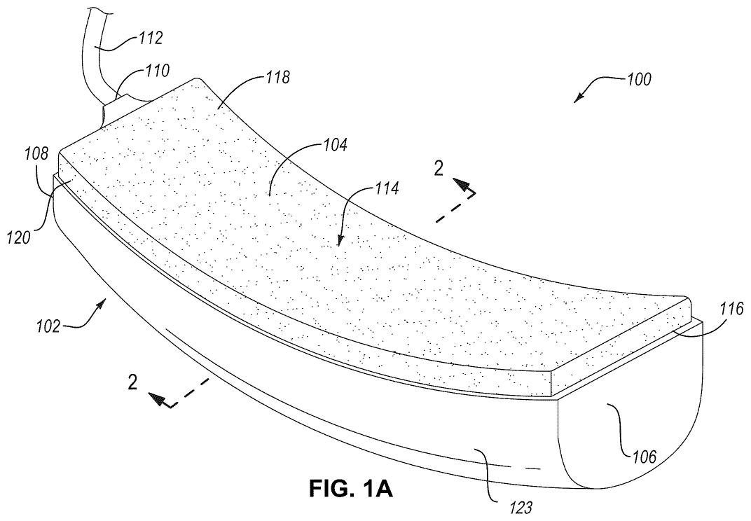

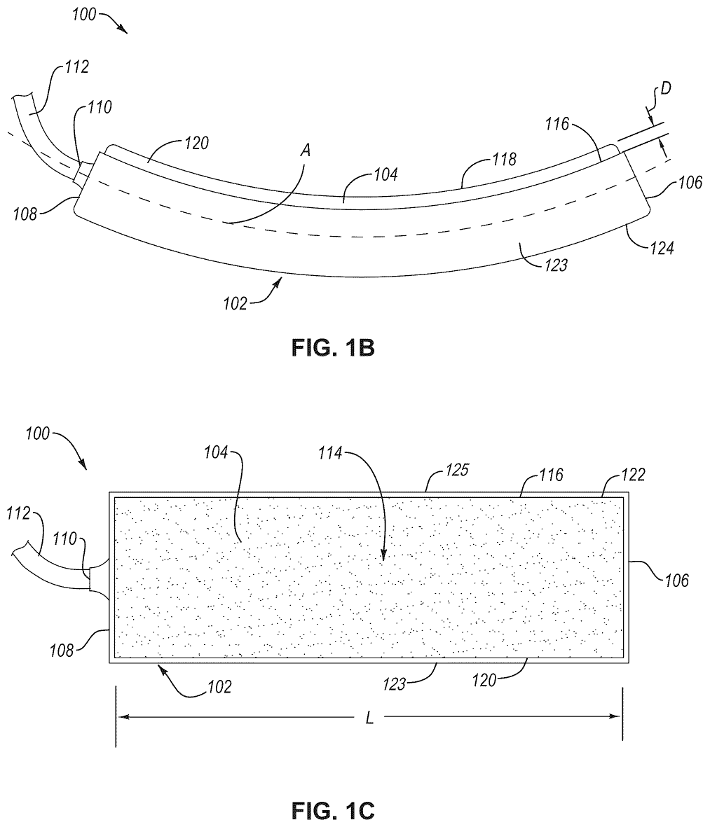

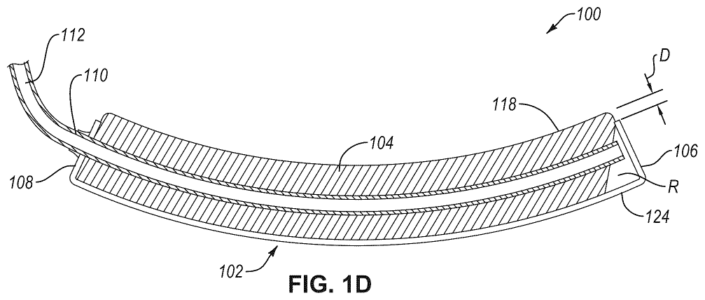

FIGS. 1A-1C are respective isometric, side, and top views of a fluid collection device 100, according to an embodiment. The fluid collection device 100 includes a fluid impermeable housing 102 and a porous material 104 disposed at least partially within the fluid impermeable housing 102. In some embodiments, the fluid impermeable housing 102 can be defined as having an elongated trapezoidal shape that can be straight or bent to exhibit a radius of curvature such that a longitudinal axis A of the fluid impermeable housing 102 bends along the length of the longitudinal axis A (see FIG. 1B). The fluid impermeable housing 102 can include a distal end 106 and a proximal end 108. The distal end 106 can form a fluid collection region or reservoir R within the fluid impermeable housing 102. The proximal end 108 can form an aperture 110 wherein a fluid conduit 112 can be partially disposed within the fluid impermeable housing 102.

The fluid impermeable housing 102 can form or otherwise define an interior cavity or internal volume 114 within the fluid impermeable housing 102. The fluid impermeable housing 102 can define an opening 116 in fluid communication with the internal volume 114. Urine and other fluids expelled by the user can be received within the opening 116 while the fluid collection device 100 is positioned at a pubic region of the user. In embodiments, the opening 116 can be elongated and extend longitudinally across the fluid impermeable housing 102. The porous material 104 can substantially fill the opening 116 and include a contact surface 118 which is positioned external to the internal volume 114 of the impermeable housing 102.

Although the embodiment depicted in FIGS. 1A-1D illustrate a single elongated opening 116, other embodiments may include two or more openings of various geometric shapes and sizes. For example, the fluid impermeable housing may form two or more rectangular shaped openings, each in fluid communication with the internal volume of the fluid impermeable housing, according to an embodiment. Accordingly, the porous material can include multiple distinct surfaces which protrude from the two or more openings.

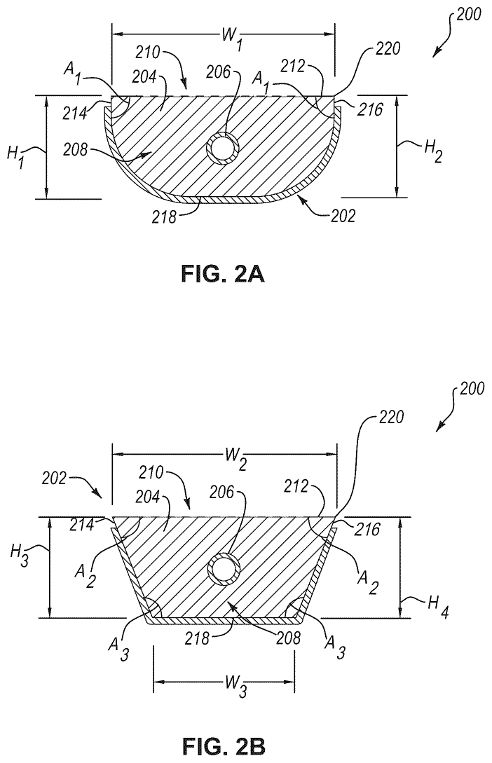

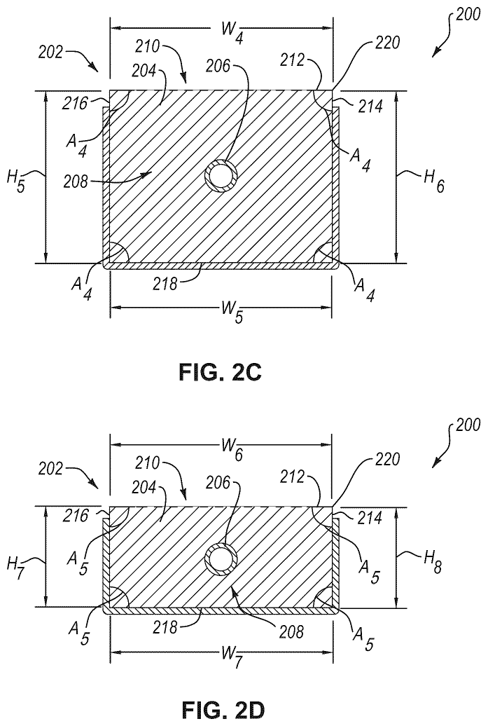

The contact surface 118 can be substantially planar and extend along a substantial length L of the fluid collection device 100. In some embodiments, the contact surface 118 is substantially flat (e.g., neither concave nor convex). The contact surface 118 can adjoin or otherwise abut one or more side walls of the porous material 104. In embodiments, the contact surface 118 can adjoin a first side wall 120 and a second side wall 122 of the porous material 104. In embodiments, each of the first and second side walls 120, 122 can be planar or substantially planar and extend from the contact surface 118 toward a back wall 124 of the fluid impermeable housing 102. In some embodiments, the fluid impermeable housing 102 may not include a back wall 124. For example, a first side wall 123 of the fluid impermeable housing 102 can be directly coupled or connected to a second side wall 125 of the fluid impermeable housing 102 without a back wall 124 indirectly coupling the first side wall 123 to the second side wall 125. Each of the first and second side walls 120, 122 of the porous material 104 can extend from the contact surface 118, for example, in an oblique, acute, or orthogonal direction relative to the contact surface 118. The position, dimensions, and other characteristics of each of the first and second side walls 120, 122 relative to the contact surface 118 will be discussed in greater detail herein in relation to the example embodiments illustrated in FIGS. 2A-2D.

The fluid impermeable housing 102 can be manufactured or formed of any suitable fluid impermeable materials, such as a fluid impermeable polymer (e.g., silicone, polypropylene, polyethylene, polyethylene terephthalate, a polycarbonate, etc.), polyurethane films, thermoplastic elastomer (TPE), rubber, thermoplastic polyurethane, another suitable material, or combinations thereof. As such, the fluid impermeable housing 102 substantially prevents the fluids from exiting the internal volume 114. The fluid impermeable housing 102 can be flexible, thereby enabling the fluid collection device 100 to bend or curve when positioned against the body of a wearer. Example fluid impermeable barriers may include, but are not limited to, a fluid impermeable barrier including at least one of Versaflex CL 2000X TPE, Dynaflex G6713 TPE, or Silpuran 6000/05 A/B silicone. The fluid impermeable housing 102 can be hydrophobic or otherwise fluid impermeable such that the fluid impermeable housing 102 does not absorb liquid, nor is liquid able to leak out of the fluid impermeable housing 102. For example, the fluid impermeable housing 102 can be a single-piece or multi-piece construction.

The porous material 104 can be at least partially disposed within the internal volume 114 formed within the fluid impermeable housing 102. For example, a majority of the porous material 104 can be disposed within the internal volume 114 while a minority of the porous material 104 can extend from the opening 116 to form the contact surface 118 which contacts or nearly contacts a urethral opening of the female patient. In other words, a portion of the porous material 104 is exposed via and projects from the opening 116 of the fluid impermeable housing 102. The exposed porous material 104 can be defined by the contact surface 118 and first and second side walls 120, 124. The exposed portion of the porous material 104 can function as a fluid outlet which wicks fluid away from the user and into the internal volume 114. While the contact surface 118 is depicted as planar or substantially planar in the figures, the contact surface 118 can form or define one or more channels, ridges, depressions, curved protrusions, or any other formation.

In examples, the porous material 104 can extend from the opening 116 a distance D (see FIGS. 1B and 1D). In other words, the contact surface 118 of the porous material 104 can be disposed a distance D from the opening 116 of the fluid impermeable housing 102. The distance D can be at least about 1 mm, between about 1 mm and about 1 cm, between about 1 cm and about 2 cm, between about 2 cm and about 3 cm, between about 3 cm and about 4 cm, or less than about 5 cm. In some embodiments, the distance D can be zero or substantially zero such that the contact surface 118 of the porous material 104 is flush or substantially flush with the opening 116 of the fluid impermeable housing 102. While the contact surface 118 is depicted as uniformly extending the distance D from the opening 116, some portions of the contact surface 118 may extend further than other portions of the contact surface 118 in some embodiments. For example, the portion of the contact surface 118 closest to the distal end 106 of the fluid impermeable housing 102 can extend further from the opening 116 than the portion of the contact surface 118 closest to the proximal end 108 of the fluid impermeable housing 102.

In embodiments, the porous material 104 can include multiple layers of material, such as fluid permeable layers having varied permeable properties. The porous material 104 can include any material that can wick fluid, for example, the porous material 104 can include fabric, such as a gauze (e.g., a silk, linen, polyester, or cotton gauze), another soft fabric (e.g., jersey knit fabric or the like), or another smooth fabric (e.g., rayon, satin, or the like). In some examples, the porous material 104 can include an open cell foam. Forming the porous material 104 from gauze, soft fabric, and/or smooth fabric can reduce chafing caused by the fluid collection device 100. The porous material 104 can additionally or alternatively be formed using one or more layers of polytetrafluoroethylene (PTFE) and/or spun plastic. In an embodiment, the porous material 104 may include spun nylon fibers with a fabric (e.g., gauze) outer permeable membrane wrapped at least around the porous material 104.

The porous material 104 can include permeable material designed to wick or pass fluid therethrough. The permeable properties referred to herein may be wicking, capillary action, diffusion, or other similar properties or processes, and are referred to herein as “permeable” and/or “wicking.” Such “wicking” may not include absorption of fluid into the porous material 104. Put another way, substantially no absorption of fluid into the porous material 104 may take place after the porous material 104 is exposed to the fluid and removed from the fluid for a time. While no absorption is desired, the term “substantially no absorption” may allow for nominal amounts of absorption of fluid into the porous material (e.g., absorbency), such as less than about 30 wt % of the dry weight of the porous material 104, less than about 20 wt %, less than about 10 wt %, less than about 7 wt %, less than about 3 wt %, less than about 2 wt %, less than about 1 wt %, or less than about 0.5 wt % of the dry weight of the porous material 104. The porous material 104 can include natural fibers. In such examples, the porous material 104 may have a coating to prevent or limit absorption of fluid into the material, such as a water repellent coating.

In embodiments, the porous material 104 can have a high permeation rate such that urine can be rapidly transported through the porous material 104. In some embodiments, the porous material 104 can include and/or have the moisture-wicking characteristic of gauze, felt, terrycloth, thick tissue paper, and/or a paper towel. In some embodiments, the porous material 104 can be soft and/or minimally abrasive such that the porous material 104 does not irritate the skin of the user. The porous material 104 can wick fluid away from the skin of the user such that moisture at or near the skin is lessened. The wicking properties of the porous material 104 can help prevent urine from leaking onto, for example, a hospital bed or a user of the fluid collection device 100. In some embodiments, the porous material 104 can be formed of fine denier polyester fibers coated with a thermoplastic water-based binder system.

The fluid conduit 112 can be formed as an elongate tube which extends from the internal volume 114 of the fluid impermeable housing 102 to outside of the fluid impermeable housing 102 and forms a fluid flow path to extract fluid from the internal volume 114. For example, the fluid conduit 112 can span substantially parallel to the longitudinal axis A of the fluid impermeable housing 102 and extend through the aperture 110 within the fluid impermeable housing 102. When suction is applied to the fluid conduit 112, the fluids disposed within the fluid impermeable housing 102 can be drawn through the fluid conduit 112 and out of the fluid collection device 100.

The fluid collection device 100 can be positioned in a pubic region of a female user. For example, the fluid collection device 100 can be disposed between the thighs and/or buttocks of the female user and abut a urethral opening of the female user's genitals. A portion of the contact surface 118 can abut or otherwise be disposed adjacent a perineum of the female user. While positioned in the pubic region of the female user, the porous material 104 can be shaped or otherwise formed to more proficiently retain the fluid collection device in a fixed position relative to the user. In embodiments, the contact surface 118, the first side wall 120, and/or the second side wall 122 can be oriented relative to each other as to provide a cross-section, which is better engaged by the patient using the fluid collection device 100 to retain the fluid collection device 100 in a fixed position relative to the patient. For example, the first and second side walls 120, 122 may be planar and orthogonal to the contact surface 118 to provide a surface area which engages with a user's thighs, buttocks, and/or genital region to retain the fluid collection device 100 in a fixed position relative to the user. Additionally, or alternatively, the contact surface 118 and the first and second side walls 120, 122 can form corners or edges which engage with a user's thighs, buttocks, and/or genital region to retain the fluid collection device 100 in a fixed position relative to the user. By incorporating a porous material 104 defined by a plurality of walls and surfaces (e.g., the contact surface 118 and the first and second side walls 120, 122), the fluid collection device 100 can be reliably retained in a desirable position relative to the patient.

As shown in FIG. 1D, the end of the fluid conduit 112 may extend through a bore in the porous material 104, such as into the fluid collection region or reservoir R. For example, an inlet of the fluid conduit 112 may extend into or be positioned in the reservoir R. In some examples, the inlet may not extend into the reservoir R. In such examples, the inlet may be disposed within the porous material 104 or at a terminal end thereof. For example, an end of the fluid conduit 112 may be coextensive with or recessed within the porous material 104.

Locating the inlet of the fluid conduit 112 at or near a location expected to be the gravimetrically low point of the internal volume 114 when worn by a user enables the fluid conduit 112 to receive more of the bodily fluids than if inlet of the fluid conduit 112 was located elsewhere and reduce the likelihood of pooling (e.g., pooling of the bodily fluids may cause microbe growth and foul odors). For instance, the bodily fluids in the porous material 104 may flow in any direction due to capillary forces. However, the bodily fluids may exhibit a preference to flow in the direction of gravity, especially when at least a portion of the porous material 104 is saturated with the bodily fluids. Accordingly, one or more of the inlet of the fluid conduit 112 or the reservoir R may be located in the fluid collection device 100 in a position expected to be the gravimetrically low point in the fluid collection device 100 when worn by a patient, such as the distal end 106.

FIGS. 2A-2D illustrate various example embodiments of fluid collection devices 200 including a fluid impermeable housing 202, a porous material 204, and a fluid conduit 206. The fluid impermeable housing 202 can be substantially similar to the fluid impermeable housing 102 previously described. For example, the fluid impermeable housing 202 can define an internal volume 208 and an opening 210 in fluid communication with the internal volume 208. The porous material 204 can include a contact surface 212, a first side wall 214, a second side wall 216, and a back wall 218.

Each of the embodiments illustrated in FIGS. 2A-2D define a particular cross-section that varies in geometry or shape. The illustrations shown in FIGS. 2A-2D are depictions of a single cross-sectional view, however, it should be appreciated that the geometry or shape of each of the respective cross-sections can vary along a length (e.g., length L of FIG. 1C) of the fluid impermeable housing 202 or remain substantially constant along the length of the fluid impermeable housing 202. For example, in embodiments, proximal and distal ends of the porous material 204 may have the cross-section depicted in FIG. 2D while a middle portion of the porous material 204 may have the cross-section depicted in FIG. 2B. While the porous material 204 is depicted as substantially filling the internal volume 208, the porous material 204 can fill a portion of the internal volume 208 in other embodiments.

The geometry or shape of the cross-sections depicted in FIGS. 2A-2D merely represent a subset of the cross-sections contemplated by the present disclosure. Accordingly, the particular geometry and shape of the cross-sectional shapes shown in FIGS. 2A-2D should not be viewed as limiting the geometry or shapes of cross-sections contemplated by this disclosure. For example, the cross-section of the porous material can form any shape, such as, a triangle, quadrilateral, pentagon, hexagon, heptagon, or any other shape comprising interconnected straight or curved lines which define a shape.

In some embodiments, the porous material 204 can form an unconventional geometric cross-section, such as, the cross-section depicted in FIG. 2A. FIG. 2A depicts an embodiment of the fluid collection device 200 including the porous material 204 forming a cross-section. The cross-section is defined by the contact surface 212 of a porous material 204 having a width W₁. The cross-section is also defined by respective heights H₁, H₂ of a first side wall 214 and a second side wall 216 of the porous material 204. The height H₁ of the first side wall 214 can be equal or about equal to the height H₂ of the second side wall 216. The first and second side walls 214, 216 can curve to connect opposite the contact surface 212, as shown in FIG. 2A. Thus, the first and second side walls 214, 216 can curve together to form a back wall 218 of the porous material 204 which is rounded or contoured. In embodiments, the first and second side walls 214, 216 can define respective angles A₁ with the contact surface 212. Each of the angles A₁ can be a right angle or about 90 degrees.

The width W₁ of the contact surface 212 can be at least about 1 cm, between about 1 cm and about 2 cm, between about 2 cm and about 3 cm, between about 3 cm and about 4 cm, between about 4 cm and about 5 cm, or less than 10 cm. The respective heights H₁, H₂ of the first side wall 214 and the second side wall 216 can be at least about 1 cm, between about 1 cm and about 2 cm, between about 2 cm and about 3 cm, between about 3 cm and about 4 cm, between about 4 cm and about 5 cm, or less than 10 cm.

FIG. 2B depicts another embodiment of the fluid collection device 200 including the fluid impermeable housing 202 forming a cross-section. The cross-section is defined by respective widths W₂, W₃ of the contact surface 212 and the back wall 218. The cross-section is also defined by respective heights H₃, H₄ of the first side wall 214 and the second side wall 216. The height H₃ of the first side wall 214 can be equal or about equal to the height H₄ of the second side wall 216. The width W₂ of the contact surface 212 can be greater than the width W₃ of the back wall 218. In embodiments, the first and second side walls 214, 216 can define respective angles A₂ with the contact surface 212 and respective angles A₃ with the back wall 218 such that the cross-section resembles a trapezoid, as shown in FIG. 2B. The respective angles A₂ can be acute and the respective angles A₃ can be obtuse, according to some embodiments.

According to the embodiment shown in FIG. 2B, the size of each respective angle A₂ can be at least about 20 degrees, between about 20 degrees and about 45 degrees, between about 45 degrees and about 70 degrees, or less than about 85 degrees. The size of each respective obtuse angle A₃ can be at least about 95 degrees, between about 95 degrees and about 120 degrees, between about 120 degrees and about 145 degrees, or less than about 160 degrees. In some embodiments, the width W₂ of the contact surface 212 can be less than or smaller than the width W₃ of the back wall 218 such that the respective angles formed by the first and second side walls 214, 216 (e.g., angles A₂) are obtuse rather than acute. Consequently, the respective angles formed by the first and second side walls 214, 216 and the back wall 218 (e.g., angles A₃) would be acute rather than obtuse. In these examples having a back wall 218 that is wider than the contact surface 212, the sizes and size range given for each of the angles A₂, A₃ described above can be reversed. For example, the angles formed by the first and second side walls 214, 216 and the contact surface 212 can range between about 95 degrees and about 160 degrees while the angles formed by the first and second side walls 214, 216 and the back wall 218 can range between about 20 degrees and about 85 degrees.

The width W₂ of the contact surface 212 can be at least about 1 cm, between about 1 cm and about 2 cm, between about 2 cm and about 3 cm, between about 3 cm and about 4 cm, between about 4 cm and about 5 cm, or less than 10 cm. Similarly, the width W₃ of the back wall 218 can be at least about 1 cm, between about 1 cm and about 2 cm, between about 2 cm and about 3 cm, between about 3 cm and about 4 cm, between about 4 cm and about 5 cm, or less than 10 cm. The respective heights H₃, H₄ of the first side wall 214 and the second side wall 216 can be at least about 1 cm, between about 1 cm and about 2 cm, between about 2 cm and about 3 cm, between about 3 cm and about 4 cm, between about 4 cm and about 5 cm, or less than 10 cm.

FIG. 2C depicts an embodiment of a fluid collection device 200 including the porous material 204 forming a cross-section. The cross-section is defined by respective widths W₄, W₅ of the contact surface 212 and the back wall 218. The cross-section is also defined by respective heights H₅, H₆ of the first side wall 214 and the second side wall 216. The height H₅ of the first side wall 214 can be equal or about equal to the height H₆ of the second side wall 216. The width W₄ of the contact surface 212 can be equal or about equal to the width W₅ of the back wall 218. Moreover, in embodiments, the respective widths W₄, W₅ of the contact surface 212 and back wall 218 can be about equal to the heights H₅, H₆ of the first and second side walls 214, 216 such that the cross-section resembles a square, as shown in FIG. 2C.

The respective widths W₄, W₅ of the contact surface 212 and the back wall 218 can be at least about 1 cm, between about 1 cm and about 2 cm, between about 2 cm and about 3 cm, between about 3 cm and about 4 cm, between about 4 cm and about 5 cm, or less than 10 cm. Similarly, the respective heights H₅, H₆ of the first side wall 214 and the second side wall 216 can be at least about 1 cm, between about 1 cm and about 2 cm, between about 2 cm and about 3 cm, between about 3 cm and about 4 cm, between about 4 cm and about 5 cm, or less than 10 cm.

While the contact surface 212, the back wall 218, and the first and second side walls 214, 216 are described herein as distinct and separate, it should be appreciated that each of the contact surface 212, the back wall 218, and the first and second side walls 214, 216 can be formed of a singular piece of machined, printed, molded or otherwise manufactured material which forms the porous material 204.

The first and second side walls 214, 216 can extend substantially orthogonal to the contact surface 212 and the back wall 218 to define respective right angles A₄ between the first and second side walls 214, 216 and the contact surface 212 and the back wall 218. While corners 220 are depicted as square edges in FIG. 2C, the corners 220 can be rounded or beveled in other embodiments. In embodiments, the angles defined by the first and second side walls 214, 216 and the contact surface 212 and the back wall 218 can be oblique angles, as depicted in FIG. 2B.

FIG. 2D depicts another embodiment of the fluid collection device 200 including the porous material 204 forming a cross-section. The cross-section is defined by respective widths W₆, W₇ of the contact surface 212 and the back wall 218. The cross-section is also defined by respective heights H₇, H₈ of the first side wall 214 and the second side wall 216. The height H₇ of the first side wall 214 can be equal or about equal to the height H₈ of the second side wall 216. The width W₆ of the contact surface 212 can be equal or about equal to the width W₇ of the back wall 218. In embodiments, the respective widths W₆, W₇ of the contact surface 212 and back wall 218 can be lesser or smaller than the heights H₇, H₈ of the first and second side walls 214, 216 such that the cross-section resembles a rectangle, as shown in FIG. 2D.

The respective widths W₆, W₇ of the contact surface 212 and the back wall 218 can be at least about 1 cm, between about 1 cm and about 2 cm, between about 2 cm and about 3 cm, between about 3 cm and about 4 cm, between about 4 cm and about 5 cm, or less than 10 cm. Similarly, the respective heights H₇, H₈ of the first side wall 214 and the second side wall 216 can be at least about 1 cm, between about 1 cm and about 2 cm, between about 2 cm and about 3 cm, between about 3 cm and about 4 cm, between about 4 cm and about 5 cm, or less than 10 cm.

The respective widths W₆, W₇ of the contact surface 212 and the back wall 218 can be correlated with the respective heights H₇, H₈ of the first side wall 214 and the second side wall 216 to define a width-to-height ratio. For example, the widths W₆, W₇ can each be 1 cm while the heights H₇, H₈ can be 2 cm to define a width-to-height ratio of 0.5 (i.e. width/height=½=0.5). The cross-section can have a width-to-height ratio of at least 0.1, between about 0.1 and about 0.3, between about 0.3 and about 0.5, between about 0.5 and about 1, between about 1 and about 2, or less than about 4.

The first and second side walls 214, 216 can extend substantially orthogonal to the contact surface 212 and the back wall 218 to define respective right angles A₅ between the first and second side walls 214, 216 and the contact surface 212 and the back wall 218. While the first and second side walls 214, 216 are depicted as extending substantially orthogonal from the contact surface 212 and the back wall 218 in FIGS. 2C and 2D, the first and second side walls 214, 216 can form respective acute angles with the contact surface 212 and form respective obtuse angles with the back wall 218 in some embodiments (as shown in FIG. 2B) or vice versa. While the corners 220 are depicted as substantially square edges in FIG. 2D, the corners 220 can be rounded or beveled in other embodiments.

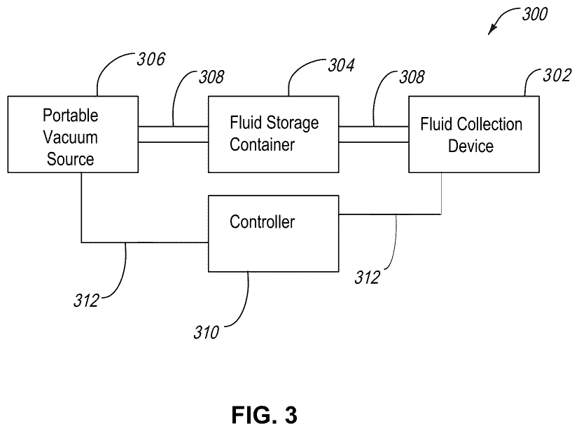

FIG. 3 is a block diagram of a system 300 for fluid collection (e.g., transporting urine away from a user), according to an embodiment. The system 300 includes a fluid collection device 302, a fluid storage container 304, a suction device or portable vacuum source 306, one or more fluid conduits 308, and a controller 310. The fluid collection device 302 may include any of the fluid collection devices described herein, such as the fluid collection device 100, 200. The fluid collection device 302, the fluid storage container 304, and the portable vacuum source 306 may be fluidly coupled to each other via one or more fluid conduits 308. The one or more fluid conduits 308 may include tubes, such as the fluid conduit 112 previously described herein. The fluid collection device 302 may be operably coupled to one or more of the fluid storage container 304 or the portable vacuum source 306 via the fluid conduit 308. Urine and/or other bodily fluids collected in the fluid collection device 302 may be removed from the fluid collection device 302 via the fluid conduit 308. For example, a first open end of the fluid conduit 308 may be coupled to the fluid collection device 302. The second open end of the fluid conduit 308 may be coupled to the portable vacuum source 306. A suction force may be introduced into an interior volume of the fluid collection device 302 via the first open end of the fluid conduit 308 responsive to a suction (e.g., vacuum) force applied at the second end of the fluid conduit 308. The suction force may be applied to the second open end of the conduit 308 by the portable vacuum source 306 either directly or indirectly.

The suction force may be applied indirectly via the fluid storage container 304. For example, the second open end of the fluid conduit 308 may be disposed within the fluid storage container 304 and an additional fluid conduit 308 may extend from the fluid storage container 304 to the portable vacuum source 306. Accordingly, the portable vacuum source 306 may apply suction to the fluid collection device 302 via the fluid storage container 304. The suction force may be applied directly via the fluid storage container 304. For example, the second open end of the fluid conduit 308 may be disposed within the portable vacuum source 306. An additional fluid conduit 308 may extend from the portable vacuum source 306 to a point outside of the fluid collection device 302, such as to the fluid storage container 304. In such examples, the portable vacuum source 306 may be disposed between the fluid collection device 302 and the fluid storage container 304.

In some embodiments, the fluid storage container 304 may include a bag (e.g., drainage bag), a bottle or cup (e.g., collection jar), or any other enclosed container for storing bodily fluids such as urine. In examples, the fluid conduit 308 may extend from the fluid collection device 302 and attach to the fluid storage container 304 at a first point therein. An additional fluid conduit 308 may attach to the fluid storage container 304 at a second point thereon and may extend and attach to the portable vacuum source 306. For example, the fluid storage container 304 may include a container fluidly coupled to a first fluid conduit 308 that is also fluidly coupled to the outlet of the fluid collection device 302. The container may be fluidly coupled to a second fluid conduit 308 that is also fluidly coupled to a portable vacuum source 306. In such examples, the portable vacuum source 306 may provide a vacuum/suction through the container to the outlet to provide suction in the interior volume of the fluid collection device. Accordingly, a vacuum (e.g., suction) may be drawn through the fluid collection device 302 via the fluid storage container 304. As the fluid (e.g., urine) is drained from the chamber, the fluid may travel through the first section of conduit to the fluid storage container where it may be retained. Fluid, such as urine, may be drained from the fluid collection device 302 using the portable vacuum source 306.

The portable vacuum source 306 may include one or more of a manual vacuum pump, and electric vacuum pump, a diaphragm pump, a centrifugal pump, a displacement pump, a magnetically driven pump, a peristaltic pump, or any pump configured to produce a vacuum. The portable vacuum source 306 may provide a vacuum or suction to remove fluid from the outlet of the fluid collection device 302. In some embodiments, the portable vacuum source 306 may be powered by one or more of a power cord (e.g., connected to a power socket), one or more batteries, or even manual power (e.g., a hand operated vacuum pump). In examples, the portable vacuum source 306 may be sized and shaped to fit outside of, on, or within the fluid collection device 302. For example, the portable vacuum source 306 may include one or more miniaturized pumps or one or more micro pumps. The portable vacuum sources 306 disclosed herein may include one or more of a switch, a button, a plug, a remote, or any other device suitable to activate the portable vacuum source 306. It should be understood that the portable vacuum sources 306 disclosed herein may provide a portable means of providing a suction or vacuum that allows use of the devices and systems herein outside of hospital or care facility environments where vacuum lines are plumbed into patient rooms or large (e.g., larger or heavier than a patient can readily carry) vacuum sources are located. For example, a portable vacuum source may be small and light enough to be carried by a user (e.g., patient) or aid (e.g., nurse) during transportation of the user.

The controller 310 can be communicatively coupled (via electrical connections 312 or wirelessly) to one or more of the fluid collection device 302 or the portable vacuum source 306. The controller 310 can direct the portable vacuum source 306 to operate when a moisture event is detected. In any of the examples, systems, or devices disclosed herein, the fluid collection device 302 may include moisture sensors (not shown) disposed inside of the fluid collection device 302. In such examples, the moisture sensor may be operably coupled to the controller 310 or directly to the portable vacuum source 306, and may provide electrical signals indicating that moisture is or is not detected within the fluid collection device 302. The moisture sensor(s) may provide an indication that moisture is present, and responsive thereto, the controller 310 or portable vacuum source 306 may direct the initiation of suction to the outlet to remove the fluid from the fluid collection device 302. Suitable moisture sensors may include capacitance sensors, volumetric sensors, potential sensors, resistance sensors, frequency domain reflectometry sensors, time domain reflectometry sensors, or any other suitable moisture sensor. In practice, the moisture sensors may detect moisture in the fluid collection device 302 and may provide a signal to the controller 310 and/or portable vacuum source 306 to activate the portable vacuum source 306. In other embodiments, the controller 310 may direct the portable vacuum source 306 to continuously operate.

While various embodiments of the fluid collection system, methods and devices have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and such modifications are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

For example, although various embodiments have been described as having particular features and/or combinations of components, other embodiments are possible having any combination or sub-combination of any features and/or components from any of the embodiments described herein. In addition, the specific configurations of the various components can also be varied. For example, the size and specific shape of the various components can be different than the embodiments shown, while still providing the functions as described herein.

1. A fluid collection device, comprising:
a fluid impermeable housing defining an internal volume, the fluid impermeable housing defining an opening in fluid communication with the internal volume, the opening in the fluid impermeable housing being defined by one or more edges of the fluid impermeable housing; and
a porous material partially disposed within and substantially filling the internal volume such that the porous material is continuous in a longitudinal direction within the internal volume and in fluid communication with the opening, the porous material including a contact surface having a first portion disposed in the internal volume and a second portion protruding from the fluid impermeable housing such that the second portion is disposed external to the internal volume and at least some of the second portion of the contact surface is spaced outward from the one or more edges of the impermeable housing defining the opening, wherein the contact surface is curved;
wherein the porous material is configured to be placed at least proximate to a urethra of a female user when the fluid collection device is disposed in a pubic region of the female user.

2. The fluid collection device of claim 1, wherein the porous material extends into the internal volume of the fluid impermeable housing from a perimeter of the contact surface through the opening.

3. The fluid collection device of claim 1, wherein the contact surface forms a curved protrusion.

4. The fluid collection device of claim 1, wherein the fluid impermeable housing includes a back wall that is curved and opposite to the opening.

5. The fluid collection device of claim 1, wherein at least some of the second portion of the contact surface spaced from the one or more edges of the fluid impermeable housing is disposed at a distance of about 1 mm to about 1 cm from the one or more edges of the fluid impermeable housing defining the opening.

6. The fluid collection device of claim 1, wherein the opening is elongated and extends longitudinally along the fluid impermeable housing.

7. The fluid collection device of claim 1, further comprising a fluid conduit disposed within the internal volume and configured to enable extraction of fluid from the internal volume.

8. The fluid collection device of claim 7, wherein the fluid impermeable housing forms an aperture at a first end of the internal volume and the fluid conduit includes a tube extending through the aperture of the fluid impermeable housing to at least proximate a second end of the internal volume.

9. The fluid collection device of claim 1, wherein the porous material includes a first side wall and a second side wall, each of the first and second side walls being substantially planar and extending into the internal volume of the fluid impermeable housing from a perimeter of the contact surface through the opening with each of the first and second side walls adjacent to opposing portions of the one or more edges of the fluid impermeable housing defining the opening.

10. A fluid collection device, comprising:
a fluid impermeable housing that is elongated and defines an internal volume and an opening that is elongated and in fluid communication with the internal volume,
a porous material partially disposed within and substantially filling the internal volume such that the porous material is continuous in a longitudinal direction within the internal volume and in fluid communication with the opening, the porous material including a contact surface that forms a curved protrusion disposed external to the internal volume such that at least at least a portion of the contact surface is spaced outward from at least a portion of the impermeable housing defining the opening, the porous material extending into the internal volume of the fluid impermeable housing from the perimeter of the contact surface through the opening,
wherein the porous material is configured to be placed at least proximate to a urethra of a female user when the fluid collection device is disposed in a pubic region of the female user.

11. The fluid collection device of claim 10, wherein:
the opening in the fluid impermeable housing is defined by a distal edge, a proximal edge, and two opposing side edges of the fluid impermeable housing extending between the distal edge and the proximal edge end;
the contact surface includes a perimeter having a distal portion, a proximal portion, and two opposing side portions generally complementary to the distal edge, the proximal edge, and the two opposing side edges of the fluid impermeable housing defining the opening; and
at least the portion of the contact surface is spaced from the plane extending along at least the two opposing side edges of the impermeable housing defining the opening.

12. The fluid collection device of claim 10, wherein at least the portion of the contact surface spaced outward from the portion of the fluid impermeable housing defining the opening is disposed at a distance of about 1 mm to about 1 cm from the portion of the impermeable housing defining the opening.

13. The fluid collection device of claim 10, wherein the opening is elongated and extends longitudinally along the fluid impermeable housing.

14. The fluid collection device of claim 10, further comprising a fluid conduit disposed within the internal volume and configured to enable extraction of fluid from the internal volume.

15. The fluid collection device of claim 14, wherein the fluid impermeable housing forms an aperture at a first end of the internal volume and the fluid conduit includes a tube extending through the aperture of the fluid impermeable housing to at least proximate a second end of the internal volume.

16. The fluid collection device of claim 10, wherein the porous material includes a first side wall and a second side wall, each of the first and second side walls being substantially planar and extending into the internal volume of the fluid impermeable housing from a perimeter of the contact surface through the opening with each of the first and second side walls adjacent to a corresponding one of the two opposing side edges.

17. The fluid collection device of claim 10, wherein the fluid impermeable housing includes a back wall that is curved and opposite to the opening.

18. A fluid collection device, comprising:
a fluid impermeable housing defining an internal volume and an opening in fluid communication with the internal volume, the opening in the fluid impermeable housing being defined by one or more edges of the fluid impermeable housing; and
a porous material partially disposed within the internal volume such that the porous material is continuous in a longitudinal direction within the internal volume and in fluid communication with the opening, the porous material including:
a contact surface forming a curved protrusion and including a perimeter generally complementary to the one or more edges of the fluid impermeable housing defining the opening, the contact surface being disposed external to and protruding outward from the internal volume such that at least some of the contact surface is spaced outward from the impermeable housing defining the opening; and
one or more walls that are substantially planar and extend into the internal volume of the fluid impermeable housing from the perimeter of the contact surface through the opening with each of the one or more walls adjacent to opposing portions of the one or more edges; and
wherein the porous material is configured to be placed at least proximate to a urethra of a female user when the fluid collection device is disposed in a pubic region of the female user.

19. The fluid collection device of claim 18, wherein at least the portion of the contact surface is disposed at a distance of about 1 mm to about 1 cm from the one or more edges of the fluid impermeable housing defining the opening.

20. The fluid collection device of claim 18, further comprising a fluid conduit disposed within the internal volume and configured to enable extraction of fluid from the internal volume, wherein the fluid impermeable housing forms a fluid reservoir at a first end of the internal volume, the fluid impermeable housing forms an aperture at a second end of the internal volume, and the fluid conduit includes a tube in fluid communication with the fluid reservoir and extending through the aperture of the fluid impermeable housing.
